(19) United States
(12) Patent Application Publication
(10) Pub. No.: US 2018/0185527 A1
(43) Pub. Date: Jul. 5, 2018

(54) CONTROL SYSTEMS FOR DISINFECTING LIGHT SYSTEMS AND METHODS OF REGULATING DISINFECTING ENERGY GENERATED BY DISINFECTING LIGHT SYSTEMS

(71) Applicant: Vital Vio, Inc., Troy, NY (US)

(72) Inventors: Jorel Lalicki, Troy, NY (US); Robert Barron, Port Washington, NY (US)

(21) Appl. No.: 15/856,971

(22) Filed: Dec. 28, 2017

Related U.S. Application Data
(60) Provisional application No. 62/440,208, filed on Dec. 29, 2016.

(51) Int. Cl. A61L 2/08

(52) U.S. Cl. CPC A61L 2/084; A61L 2202/11; A61L 2202/14

(57) ABSTRACT

Control systems for disinfecting light systems and methods of regulating disinfecting energy generated by disinfecting systems are disclosed. The control system may include a first sensor and a second sensor positioned within a space illuminated by the disinfecting light system. The first sensor may measure an amount of disinfecting energy provided to the space by the disinfecting light system, and the second sensor may detect an environmental characteristic of the space. Additionally, the control system may include a controller operably coupled to the first and second sensor. The controller may regulate the disinfecting energy generated by the disinfecting light system by adjusting the amount of disinfecting energy provided to the space by the disinfecting light system in response to the amount of disinfecting energy provided to the space by the disinfecting light system measured by the first sensor, and/or the environmental characteristic detected by the second sensor.

### CROSS-REFERENCE TO RELATED APPLICATIONS

This application claims the benefit of U.S. Provisional Application Ser. No. 62/440,208, filed 29 Dec. 2016, which is hereby incorporated by reference herein.

### BACKGROUND OF THE INVENTION

The disclosure relates generally to illumination, and more particularly, to control systems for a disinfecting light emitting diode (LED) lighting system and methods of regulating disinfecting energy generated by disinfecting LED lighting systems.

Light-emitting devices are a primary requirement in most indoor occupied environments to provide illumination of the area, of tasks being completed in the area, and of the area's occupants and objects. Alternative light sources have been created with additional performance factors in mind that utilize emitted light in different manners. Lighting fixtures and devices for horticulture, health, warmth, and disinfection have been demonstrated. In addition to being tuned for luminous efficacy of radiation, these lighting fixtures and devices are tuned to provide increased outputs of certain regions of radiation to accomplish the additional performance factor. In these lighting fixtures and devices that emit light for multiple functions, the light emissions can be balanced to achieve an acceptable level of each function. One of the functions can be general illumination (e.g., when the multiple-function lighting fixtures and devices are used in spaces occupied by humans), in which case, achieving a relatively high luminous efficacy of the emitted light is balanced not only against achieving desirable color characteristics of the emitted light, but also of achieving the one or more other functions to an acceptable or desired level. New laws and regulations around energy efficiency in residential and commercial spaces means that these multiple function light sources must also have control systems to balance energy efficiency in addition to their desired effects.

One new function of lighting is disinfecting, e.g. using blue light in combination with other light to emit what is perceived as white light. Unlike ultraviolet light (UV), white disinfecting light can be used on 24 hour/7 days without harming the occupants of a room. UV systems require extensive safety measures to prevent accidental exposure or unknown occupants and have emergency shut off switches in situations of accidental occupancy. UV systems include remote controlled robots and lockable rooms, which can only be used when a room is not occupied, which is not always feasible. Disinfecting white light does not require such safety features.

### BRIEF DESCRIPTION OF THE INVENTION

A first aspect of the disclosure provides a control system for a disinfecting light system. The control system includes: a first sensor positioned within a space illuminated by the disinfecting light system, the first sensor measuring an amount of disinfecting energy provided to the space by the disinfecting light system; a second sensor positioned within the space illuminated by the disinfecting light system, the second sensor detecting an environmental characteristic of the space; and a controller operably coupled to the first sensor and the second sensor, the controller regulating the disinfecting energy generated by the disinfecting light system by performing processes including: adjusting the amount of disinfecting energy provided to the space by the disinfecting light system in response to at least one of: the amount of disinfecting energy provided to the space by the disinfecting light system measured by the first sensor, or the environmental characteristic detected by the second sensor.

A second aspect of the disclosure provides a control system for a disinfecting light system. The control system includes: a first sensor positioned within a space including the disinfecting light system, the first sensor sensing a bacterial load of the space; a second sensor positioned within the space including the disinfecting light system, the second sensor detecting an environmental characteristic of the space; and a controller operably coupled to the first sensor and the second sensor, the controller regulating a disinfecting energy generated by the disinfecting light system by performing processes including: adjusting the amount of disinfecting energy provided to the space by the disinfecting light system in response to at least one of: the bacterial load of the space sensed by the first sensor, or the environmental characteristic detected by the second sensor.

A third aspect of the disclosure provides a control system for a disinfecting light system. The control system includes: a first sensor positioned within a first space illuminated by a first disinfecting light fixture of the disinfecting light system, the first sensor measuring an amount of disinfecting energy provided to the first space by the first disinfecting light fixture; a second sensor positioned within the first space illuminated by the first disinfecting light fixture, the second sensor sensing a bacterial load of the first space; and a controller operably coupled to the first sensor and the second sensor, the controller regulating the disinfecting energy generated by the first disinfecting light fixture by performing processes including: adjusting the amount of disinfecting energy provided to the first space by the first disinfecting light fixture in response to at least one of: the amount of disinfecting energy provided to the first space by the first disinfecting light fixture measured by the first sensor, or the bacterial load of the first space sensed by the second sensor.

A fourth aspect of the disclosure provides a method of regulating a disinfecting energy generated by a disinfecting light system. The method includes: comparing a measured amount of disinfecting energy provided to a space by the disinfecting light system to a disinfecting energy threshold; comparing a preferred amount of disinfecting energy associated with a detected, environmental characteristic to the measured amount of disinfecting energy provided to the space by the disinfecting light system; and adjusting the amount of disinfecting energy provided to the space by the disinfecting light system in response to at least one of: determining the measured amount of disinfecting energy provided to the space by the disinfecting light system does not meet the disinfecting energy threshold, or determining the measured amount of disinfecting energy provided to the space by the disinfecting light system does not meet the preferred amount of disinfecting energy associated with the detected, environmental characteristic.

The illustrative aspects of the present disclosure are designed to solve the problems herein described and/or other problems not discussed.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 shows a schematic view of an illustrative environment including a disinfecting light system and a control system, according to embodiments of the disclosure.

FIG. 2 shows a flow chart of example processes for regulating disinfecting energy generated by a disinfecting light system within a space, according to embodiments of the disclosure.

FIG. 3 shows a schematic view of a disinfecting light system including a control system, according to embodiments of the disclosure.

FIG. 4 shows a schematic view of a control system including a controller that regulates disinfecting energy generated by a disinfecting light system, according to embodiments of the disclosure.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

As an initial matter, in order to clearly describe the current disclosure it will become necessary to select certain terminology when referring to and describing relevant components within the disclosure. When doing this, if possible, common industry terminology will be used and employed in a manner consistent with its accepted meaning. Unless otherwise stated, such terminology should be given a broad interpretation consistent with the context of the present application and the scope of the appended claims. Those of ordinary skill in the art will appreciate that often a particular component may be referred to using several different or overlapping terms. What may be described herein as being a single part may include and be referenced in another context as consisting of multiple components. Alternatively, what may be described herein as including multiple components may be referred to elsewhere as a single part.

As indicated above, the disclosure relates generally to illumination, and more particularly, to control systems for a disinfecting light emitting diode (LED) lighting system and methods of regulating disinfecting energy generated by disinfecting LED lighting systems.

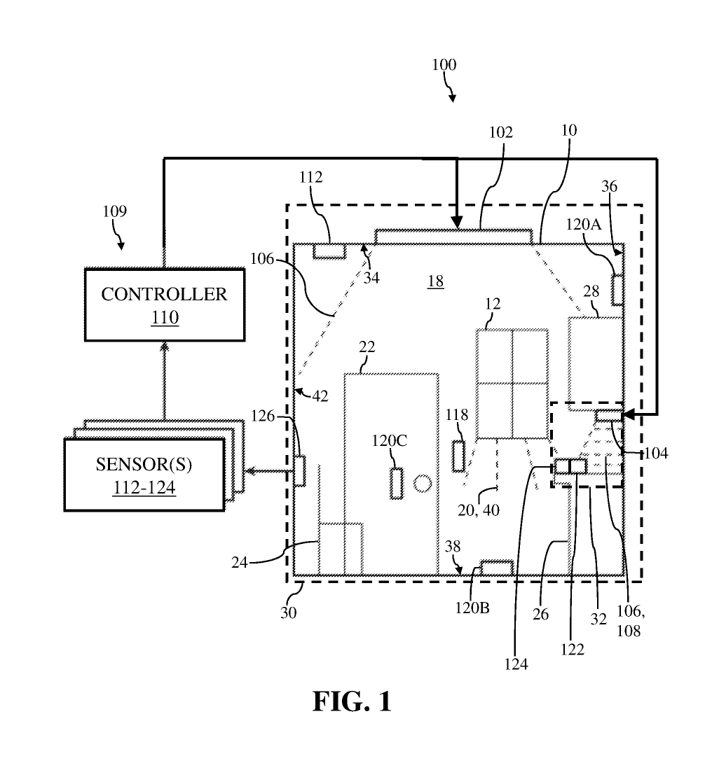

These and other embodiments are discussed below with reference to FIGS. 1-4. However, those skilled in the art will readily appreciate that the detailed description given herein with respect to these Figures is for explanatory purposes only and should not be construed as limiting.

FIG. 1 shows a schematic view of an illustrative environment including a disinfecting light system and a control system. Specifically, FIG. 1 shows environment 10 and a disinfecting light system 100 that may be at least partially positioned within and/or may interact with environment 10. In the non-limiting example shown in FIG.1, environment 10 may be a room within a building. As discussed herein, disinfecting light system 100 may illuminate environment 10, as well as provide disinfecting energy within environment 10 in order to disinfect environment 10. It is understood that the term “environment” and “room” may be used interchangeably when discussing the non-limiting examples herein. Additionally, although shown as only a single room, it is understood that environment 10 may include a plurality of rooms and/or distinct areas that include disinfecting light system 100.

As shown in the non-limiting example of FIG. 1, environment 10 may include at least one of the items and/or objects included therein. Specifically, and at least partially dependent on the type of environment 10 (e.g., room), environment 10 may include a plurality of items and/or objects positioned within environment 10. For example, environment 10 formed as a room may include a window 12 formed in one of a plurality of walls 18 of environment. As such in FIG. 1, the window may provide an opening to environment 10 which may allow sunlight or natural light 20 to enter and/or be emitted into environment 10. Additionally, in non-limiting examples, environment 10 may also include a door 22 to allow user(s) (not shown) to access environment 10 and/or the items or objects positioned therein. Additionally, environment 10 may also include a chair 24, desk or workstation 26 (hereafter, “workstation 26”), and cabinets 28. In a non-limiting example, workstation 26 may be a “clean” or “sterile” workstation or area that may be used for specific, sterile procedures and/or processing (e.g., sterile table for microchip inspection). As discussed herein, the items and/or objects (e.g., window 12, chair 24, workstation 26, and so on) may be accounted for when regulating the disinfecting energy provided to environment 10 by disinfecting light system 100. Additionally, characteristics and/or properties of environment 10 may also be accounted for and/or may affect the regulation of the disinfecting energy provided to environment 10, as discussed herein. For example, and as discussed in detail herein, the color of paint on walls 18/door 22, the amount of sun exposure for environment 10 based on window 12, the size of environment 10, and so on, may be accounted for and/or may affect the regulation of the disinfecting energy provided to environment 10 by disinfecting light system 100.

Environment 10 may include one or more spaces defined therein. For example, environment 10 may include and/or be “divided” into a plurality of distinct spaces 30, 32. Specifically, and as shown in the non-limiting example of FIG. 1, environment 10 (e.g., room) may include a first space 30 and a second space 32. In this non-limiting example, second space 32 may be included within first space 30. However, and as discussed herein, second space 32 may be defined as distinct and/or unique from first space 30 by disinfecting light system 100. That is, the spaces 30, 32 of environment 10 may be based on and/or may be defined by, at least in part, disinfecting light system 100 of environment 10 and its various components (e.g., light fixtures), as discussed herein. Additionally, or alternatively, the plurality of spaces 30, 32 of environment 10 may be based on and/or may be defined by, at least in part, items and/or objects of environment 10 (e.g., sterile workstation 26), and/or characteristics and/or properties of environment 10, as discussed herein.

The number of spaces 30, 32 shown in FIG. 1 and included within environment 10 are merely illustrative. As such, although two spaces 30, 32 are shown and discussed herein, it is understood that environment 10 may include more or less spaces. In other non-limiting examples where environment 10 includes a plurality of rooms, spaces of environment 10 may be defined as each individual room making up environment 10. Additionally, or alternatively in the non-limiting examples, each of the plurality of rooms of environment 10 may include one or more spaces similar to those discussed herein with respect to FIG. 1.

Disinfecting light system 100 included and/or operating within environment 10 may include at least one disinfecting light fixture 102, 104. Disinfecting light fixture(s) 102, 104 may be positioned within, exposed to, illuminate and/or provide (light) energy to environment 10. That is, and as discussed herein, disinfecting light fixture(s) 102, 104 may be positioned within and/or exposed to environment 10 to provide illuminating light and/or disinfecting energy to environment 10. As shown in the non-limiting example of FIG. 1, a first disinfecting light fixture 102 may be positioned within environment 10 and may be coupled to a ceiling 34 of environment 10. Additionally, a second disinfecting light fixture 104 may be positioned within environment 10 and may be coupled to cabinets 28, adjacent workstation 26. As discussed herein, the plurality of disinfecting light fixture(s) 102, 104 within environment 10 may define, at least in part, space(s) 30, 32 of environment 10. Specifically, the position of each of disinfecting light fixture(s) 102, 104 within environment 10 and/or the area of environment in which the plurality of disinfecting light fixtures(s) 102, 104 may illuminate and/or provide disinfecting energy may, at least in part, define space(s) 30, 32 of environment 10. First disinfecting light fixture 102 coupled to ceiling 34 of environment 10 may illuminate and/or provide disinfecting energy to substantially all of environment 10 (e.g., room). Therefore, the light emitted by first disinfecting light fixture 102 may define, at least in part, first space 30. Additionally, second disinfecting light fixture 104 coupled to cabinet 28 of environment 10 may illuminate and/or provide disinfecting energy to workstation 26. As such, the light emitted by second disinfecting light fixture 104 may define, at least in part, second space 32.

The plurality of disinfecting light fixture(s) 102, 104 of disinfecting light system 100 may be any suitable light fixture, component, or assembly that is capable of providing a spectral range of light energy, illumination, and/or illuminating light, as well as, disinfecting energy to environment 10. Additionally, the plurality of disinfecting light fixture(s) 102, 104 may be any suitable light fixture, component, or assembly that is capable of providing only illuminating light, only disinfecting energy, or both illuminating light and disinfecting energy simultaneously. Additionally, the plurality of disinfecting light fixture(s) 102, 104 may be any suitable light fixture, component, or assembly that is capable of switching between providing only illuminating light, only disinfecting energy, or both illuminating light and disinfecting energy simultaneously. For example, the plurality of disinfecting light fixture(s) 102, 104 of disinfecting light system 100 may include light fixtures similar to those described in U.S. Pat. No. 9,333,274, U.S. Pat. No. 9,439,989, and U.S. Pat. Pub. No. 2017/0030555 the entirety of which is hereby incorporated herein by reference. In the non-limiting example shown in FIG. 1, first disinfecting light fixture 102 may emit only light energy and/or illuminating light 106 (hereafter, “illuminating light 106”) to first space 30 of environment 10, while second disinfecting light fixture 104 may emit both illuminating light 106, as well as, disinfecting energy 108 to second space 32 of environment 10. As discussed herein, the output (e.g., illuminating light 106, disinfecting energy 108) of each of the plurality of disinfecting light fixture(s) 102, 104 may be based on sensed or measured characteristics of space(s) 30, 32 of environment 10, characteristics and/or properties of space(s) 30, 32 of environment 10, and/or predetermined information (e.g., scheduled outputs) for environment 10.

The number of disinfecting light fixture(s) 102, 104 included within environment 10, as shown in the non-limiting example of FIG. 1, is understood to be illustrative. As such, although disinfecting light system 100 is shown to include two disinfecting light fixture(s) 102, 104, it is understood that disinfecting light system 100 may include more or less disinfecting light fixture(s). Additionally, the position of disinfecting light fixture(s) 102, 104 included within environment 10, as shown in the non-limiting example of FIG. 1, is understood to be illustrative. Disinfecting light fixture(s) 102, 104 of disinfecting light system 100 may be positioned anywhere within, adjacent to, and/or exposed to environment 10 to provide illuminating light 106 and/or disinfecting energy 108 to a defined space within environment 10, as discussed herein. Furthermore, and as discussed in detail in U.S. Pat. No. 9,333,274, U.S. Pat. No. 9,439,989, and U.S. Pat. Pub. No. 2017/0030555 incorporated herein by reference, illuminating light 106 may generate visible light energy within the spectral range of approximately 380 nanometers (nm) to approximately 750 nm, and disinfecting energy 108 may be a disinfecting energy within the spectral range of approximately 380 nm to approximately 420 nm (e.g., 405 nm). That is, illuminating light 106 may include visible light energy within a spectral range that may illuminate and/or provide light to space(s) 30, 32 of environment 10. Additionally, disinfecting energy 108 generated by disinfecting light fixture(s) 102, 104 (see, second disinfecting light fixture 104) may include disinfecting energy within the spectral range that may alter, adjust, and/or control the bacterial load, bioburden, and/or microbial load (e.g., disinfect, or sterilize) within space(s) 30, 32 receiving disinfecting energy 108. In another non-limiting example, disinfecting energy 108 may include ultraviolent (UV) light having disinfecting properties and including a spectral range of approximately 100 nm to approximately 400 nm.

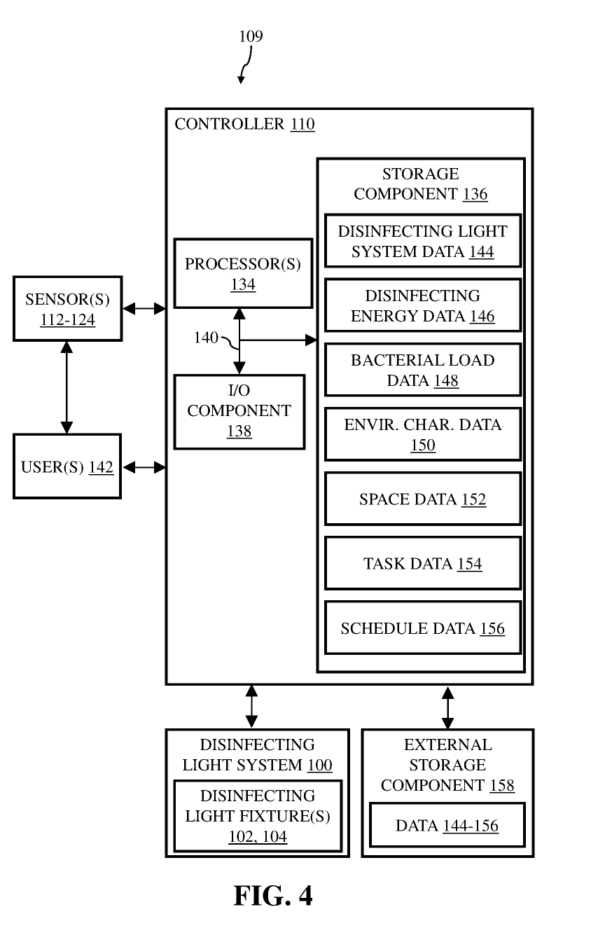

As shown in FIG. 1, disinfecting energy system 100 can include a control system 109 including at least one controller 110 configured to control operation of disinfecting light fixture(s) 102, 104. That is, controller 110 of control system 109 may be configured to regulate illuminating light 106 and disinfecting energy 108 provided to space(s) 30, 32 of environment 10 via disinfecting light fixture(s) 102, 104. Controller 110 can be hard-wired and/or wirelessly connected to, operably coupled to, and/or in communication with disinfecting light fixture(s) 102, 104 via any suitable electronic and/or mechanical communication component or technique. Controller 110, and its various components discussed herein (see, FIG. 4), may be a single stand-alone system that functions separate from another system (e.g., computing device) (not shown) that may control and/or adjust operations or functions of other portions of environment 10 (e.g., HVAC system). Alternatively, controller 110 may be integrally formed within, in communication with and/or formed as a part of a larger control system (e.g., computing device) (not shown) that may control and/or adjust operations or functions of environment 10. For example, controller 110 of control system 109 may be configured or formed as a microcontroller or similarly embedded system on a chip (SOC) component running a real-time operating system (RTOS).

Additionally, in the non-limiting example shown in FIG. 1, control system 109 for disinfecting light system 100 may also include one or more sensors 112, 118, 120A, 120B, 120C, 122, 124 operably coupled to and/or in communication with controller 110 for aiding controller 110 in controlling the operation of disinfecting light fixture(s) 102, 104. As discussed herein, controller 110 may utilize data, real-time information, and/or environment characteristics of space(s) 30, 32 of environment 10, as determined by sensor(s) 112, 118, 120A, 120B, 120C, 122, 124, to control the operation of disinfecting light fixture(s) 102, 104 to ultimately regulate illuminating light 106 and disinfecting energy 108 provided to space(s) 30, 32 of environment 10.

As shown in FIG. 1, controller 110 of control system 109 may be operably coupled to, in electrical and/or mechanical communication with sensor(s) 112, 118, 120A, 120B, 120C, 122, 124 positioned throughout environment 10 (e.g., one shown). Additionally, and as shown in the non-limiting example of FIG. 1, controller 110 may be wirelessly connected to, and/or in communication with sensor(s) 112, 118, 120A, 120B, 120C, 122, 124. Sensor(s) 112, 118, 120A, 120B, 120C, 122, 124 may be positioned in various locations and/or throughout environment 10, and more specifically space(s) 30, 32. The position and/or location of sensor(s) 112, 118, 120A, 120B, 120C, 122, 124 within space(s) 30, 32 of environment 10 may be dependent, at least in part, on the type of sensor, and/or the data, information, and/or characteristic of space(s) 30, 32 the sensor is measuring, detecting, and/or sensing. Sensor(s) 112, 118, 120A, 120B, 120C, 122, 124 in communication with controller 110 of control system 109 may be any suitable sensor or device configured to detect and/or determine data, information, and/or characteristics relating to environment 10. For example, and as discussed in detail herein, sensor(s) 112, 118, 120A, 120B, 120C, 122, 124 positioned within space(s) 30, 32 may be any suitable sensor configured to detect, measure, sense, and/or determine an amount of disinfecting energy 108 provided to space(s) 30, 32 by disinfecting light fixture(s) 102, 104, a bacterial load for space(s) 30, 32, and/or environmental characteristics (e.g., occupancy, daylight) for space(s) 30, 32.

In the non-limiting example shown in FIG. 1, environment 10 may include a first sensor 112. Specifically, first space 30 of environment 10 may include first sensor 112 positioned therein and in (wireless) communication with and/or operably connected to controller 110 of control system 109. In the non-limiting example, first sensor 112 may be positioned on and/or coupled to ceiling 34 within first space 30. First sensor 112 may be configured as any suitable sensor capable of measuring an amount of disinfecting energy 108 provided to space 30 by first disinfecting light fixture 102 of disinfecting light system 100. For example, first sensor 112 of disinfecting light system 100 may include or be formed as a spectrometer, a photodiode, a watt meter, or any other suitable sensor that may be capable of measuring and/or detecting the amount of disinfecting energy 108 provided to first space 30 by first disinfecting light fixture 102.

The amount of disinfecting energy 108 provided to first space 30 by first disinfecting light fixture 102, as measured by first sensor 112, may be provided or transmitted to controller 110 to aid in controller's regulation of disinfecting energy 108 generated by first disinfecting light fixture 102 of disinfecting light system 100. As discussed herein, controller 110 may compare the measured amount of disinfecting energy 108 provided to first space 30 by first disinfecting light fixture 102 to a disinfecting energy threshold, and may adjust the amount of disinfecting energy 108 provided to space 30 by adjusting the output of first disinfecting light fixture 102. Although shown as being coupled to ceiling 34 within first space 30 of environment 10, it is understood that first sensor 112 may be positioned anywhere within first space 30 so long as first sensor 112 is capable of measuring the amount of disinfecting energy 108 provided to first space 30 by first disinfecting light fixture 102.

As shown in FIG. 1, environment 10 may also include a second sensor 118. Specifically, first space 30 of environment 10 may include second sensor 118 positioned therein and in (wireless) communication with and/or operably connected to controller 110 of control system 109. In the non-limiting example, second sensor 118 may be positioned on and/or coupled to wall 18 within first space 30. Second sensor 118 may be configured as any suitable sensor capable of sensing a bacterial load of space 30. More specifically, second sensor 118 may be any suitable sensor capable of sensing bacterial load, bioburden, and/or microbial load within space 30 of environment 10. For example, second sensor 118 of disinfecting light system 100 may include or be formed as an optical sensor, oxygen-depletion sensor, luminometer, or any other suitable sensor that may be capable of sensing a bacterial load within first space 30. In non-limiting examples, second sensor 118 may sense the bacterial load of first space 30 by measuring the bacterial load of the air within first space 30, and/or measuring the bacterial load on a surface of an object or item (e.g., window 12, wall 20, door 22, chair 24, and so on) positioned within first space 30.

In other non-limiting examples, the bacterial load of first space 30 may be based on a correlated measurement. The correlated measurement may be a calculated or determined bacterial load based on collected data that may be correlated to a bacterial load measurement. That is, data collected, measured, determined, and/or sensed by second sensor 118 may be provided to controller 110, which in turn may process and/or utilize the data from second sensor 118 to calculate or determined the bacterial load forming the correlated measurement. In non-limiting examples, the data collected by second sensor 118 may not be data including and/or pertaining directly to bacteria, microbial, and/or bioburden data, but rather may be data that can be utilized to calculate or determined the bacterial load, as discussed herein.

The bacterial load of first space 30 may change based on changes within first space 30. For example, the bacterial load of first space 30 may increase as a result of increased room occupancy by users, when new items or objects are introduced to first space 30 of environment 10, and/or over a period of time where first disinfecting light fixture 102 is not providing disinfecting energy 108 to first space 30. The bacterial load of space 30, sensed by second sensor 118, may be provided or transmitted to controller 110 to aid in controller's regulation of disinfecting energy 108 generated by first disinfecting light fixture 102 of disinfecting light system 100. As discussed herein, controller 110 may compare the sensed bacterial load of first space 30 to a bacterial load threshold, and may adjust the amount of disinfecting energy 108 provided to space 30 by adjusting the output of first disinfecting light fixture 102. That is, the bacterial load sensed by second sensor 118 within first space 30 may be directly affected and/or impacted by the amount of disinfecting energy 108 provided to space by first disinfecting light fixture 102. Although shown as being coupled to wall 18 within space 30 of environment 10, it is understood that second sensor 118 may be positioned anywhere within space 30 so long as second sensor 118 is capable of sensing the bacterial load of first space 30.

First space 30 of environment 10 may also include at least one additional, third sensor 120A, 120B, 120C positioned therein and in (wireless) communication with and/or operably connected to controller 110 of control system 109. In the non-limiting example, control system 109 may include a plurality of third sensors 120A, 120B, 120C positioned throughout first space 30 of environment 10. Each of the plurality of third sensors 120A, 120B, 120C may be configured as environmental characteristic sensors, and/or may be sensors configured to measure or detect environmental characteristics of first space 30 of environment 10. As discussed herein, a preferred amount of disinfecting energy for and/or to be provided to first space 30 may be associated with the environmental characteristics detected by third sensors 120A, 120B, 120C within first space 30. Additionally, controller 110 may compare the measured amount of disinfecting energy 108 within first space 30 (e.g., first sensor) with the preferred amount of disinfecting energy associated with detected environmental characteristics of first space 30, and may adjust the amount of disinfecting energy 108 provided to space 30 by adjusting the output of first disinfecting light fixture 102. Also discussed herein, each of the environmental characteristics detected by third sensors 120A, 120B, 120C may include a preferred amount or level of illuminating light that may be associated with the detected environmental characteristic(s). In the non-limiting example shown in FIG. 1, and discussed herein, the plurality of third sensors 120A, 120B, 120C configured to detect environmental characteristics of first space 30 may all be the distinct types of sensors and/or may detect distinct environmental characteristics of first space 30. In another non-limiting example, the plurality of third sensors 120A, 120B, 120C may all be the same type of sensor and/or may detect the same environmental characteristics of first space 30.

Third sensor 120A may be positioned on and/or coupled to a wall 36 within first space 30. Additionally, third sensor 120A may be coupled to wall 36, above cabinet 28 included within first space 30. Third sensor 120A may be configured as any suitable sensor capable of measuring or detecting an occupancy level (e.g., environmental characteristic) for first space 30. The detected occupancy level for first space 30 may include whether or not first space 30 is being occupied and/or includes a user(s) positioned therein, the number of users that may occupy first space 30 and/or a (real-time) change in occupancy for first space 30. In non-limiting examples, third sensor 120A of control system 109 may include or be formed as an infrared sensor, an automated camera system (e.g., image processing with camera based sensors), radar sensor, Lidar sensor, audio sensor, tomographic motion sensor, microwave sensor, ultrasonic sensor, or any other suitable sensor that may be capable of detecting an occupancy level of first space 30.

The occupancy level of first space 30, as detected by third sensor 120A, may be provided or transmitted to controller 110 to aid in controller's regulation of disinfecting energy 108 generated by first disinfecting light fixture 102 of disinfecting light system 100. As discussed herein, controller 110 may receive the occupancy level of first space 30 from third sensor 120A, along with a preferred amount of disinfecting energy associated with the occupancy level of first space 30. Additionally, controller 110 may compare the measured amount of disinfecting energy 108 of first space 30 (e.g., first sensor 112) with the preferred amount of disinfecting energy associated with the occupancy level of first space 30 detected by third sensor 120A, and may adjust the amount of disinfecting energy 108 provided to space 30 by adjusting the output of first disinfecting light fixture 102. Furthermore, and similar to the preferred amount of disinfecting light, controller 110 may adjust the amount of illuminating light 106 provided to space 30 by adjusting the output of first disinfecting light fixture 102 based on the preferred amount of illuminating light that may be associated with the detected, occupancy level of first space 30. Although shown as being coupled to wall 36 within first space 30 of environment 10, it is understood that third sensor 120A may be positioned anywhere within first space 30 so long as third sensor 120A is capable of detecting the occupancy level of first space 30.

Third sensor 120B may be positioned on and/or coupled to floor 38 of environment 10. Specifically, third sensor 120B may be coupled to floor 38 with first space 30, substantially adjacent, aligned with, below and/or within proximity of window 12 included within first space 30 of environment 10. Additionally, as shown in FIG. 1, third sensor 120B may be positioned substantially below, aligned with, and/or within proximity of first disinfecting light fixture 102 of disinfecting light system 100. Third sensor 120B may be configured as any suitable sensor capable of detecting an amount of natural light in first space 30. That is, third sensor 120B may be configured as a daylight sensor that may sense an amount of natural light 20 included within first space 30. In non-limiting examples, third sensor 120B of control system 109 may include or be formed as a spectrometer, a photodiode, a watt meter, or any other suitable sensor that may be capable of sensing an amount of natural light 20 within first space 30. The amount of natural light 20 of first space 30, as sensed by third sensor 120B, may be provided or transmitted to controller 110 to aid in controller's regulation of disinfecting energy 108 generated by first disinfecting light fixture 102 of disinfecting light system 100. As discussed herein, controller 110 may receive the amount of natural light 20 of first space 30 from third sensor 120B, along with a preferred amount of disinfecting energy associated with the detected amount of natural light 20 of first space 30. Additionally, controller 110 may compare the measured amount of disinfecting energy 108 of first space 30 (e.g., first sensor 112) with the preferred amount of disinfecting energy associated with the amount of natural light 20 of first space 30 detected by third sensor 120B, and may adjust the amount of disinfecting energy 108 provided to space 30 by adjusting the output of first disinfecting light fixture 102. Furthermore, and similar to the preferred amount of disinfecting light, controller 110 may adjust the amount of illuminating light 106 provided to space 30 by adjusting the output of first disinfecting light fixture 102 based on the preferred amount of illuminating light that may be associated with the detected, natural light 20 of first space 30.

Additionally, the amount of natural light 20 sensed by third sensor 120B may also include a known, calculated, predetermined, and/or measurable amount of natural disinfecting energy 40 (e.g., spectral energy of approximately 405 nm), which may be provided to first space 30. In one non-limiting example, third sensor 120B of control system 109 may be configured to measure an amount of natural disinfecting energy 40 provided to first space 30 along with natural light 20. In another non-limiting example, the amount of natural disinfecting energy 40 from natural light 20 may be calculated or determined based on a variety of factors including, but not limited to, the time of day, the date, the position of first space 30 and/or window 12 (e.g., facing east), and characteristics of window 12 (e.g., double-pane, blue light blocker, tinted, and so on). In a non-limiting example, controller 110 of control system 109 may receive the measured amount of natural disinfecting energy 40, or determine the amount of natural disinfecting energy 40, provided to first space 30 via natural light 20 sensed by third sensor 120B, and may adjust the amount of disinfecting energy 108 provided to space 30 by adjusting the output of first disinfecting light fixture 102. Although shown as being positioned on floor 38 of first space 30, it is understood that third sensor 120B may be positioned anywhere within first space 30 so long as third sensor 120B is capable of sensing the amount of natural light 20 for first space 30.

As shown in the non-limiting example of FIG. 1, third sensor 120C of control system 109 may be positioned within first space 30 of environment 10. Specifically, third sensor 120C may be coupled to door 22 within first space 30. Third sensor 120C may be configured as any suitable sensor capable of identifying at least one task being carried out in first space 30. That is, third sensor 120C may be a task-identifying sensor that may detect, sense, and/or identify task(s) being carried out and/or performed within first space 30 of environment 10. The identified task being carried out and/or performed within the first space 30 may require a predetermined or preferred amount of illuminating light 106 and/or disinfecting energy 108 to be provided to first space 30 by first disinfecting light fixture 102 when performing the task. In non-limiting examples, third sensor 120C of control system 109 may include or be formed as a camera sensor (e.g., image processing with camera based sensors) and/or a scanner sensor that may detect certain work pieces and/or users associated with a predetermined task are positioned or located within first space 30. Additionally in another non-limiting, third sensor 120C may be formed as a component-detection sensor, which may be configured to identify when an object, and/or item (e.g., microscope (not shown)) of first space 30 that is associated with and/or used specifically for a certain task is being utilized within first space 30.

Controller 110 may identify that a task(s) is being carried out in first space 30, via third sensor 120C, and may adjust the amount of disinfecting energy 108 provided to space 30 by adjusting the output of first disinfecting light fixture 102. That is, controller 110 may receive the task(s) being carried out in first space 30, as identified by third sensor 120C, along with a preferred amount of disinfecting energy associated with the identified task being carried out in first space 30. Additionally, controller 110 may compare the measured amount of disinfecting energy 108 of first space 30 (e.g., first sensor 112) with the preferred amount of disinfecting energy associated with the identified task(s) of first space 30, identified by third sensor 120C, and may adjust the amount of disinfecting energy 108 to space 30 by adjusting the output by first disinfecting light fixture 102. Furthermore, and similar to the preferred amount of disinfecting light, controller 110 may adjust the amount of illuminating light 106 provided to space 30 by adjusting the output of first disinfecting light fixture 102 based on the preferred amount of illuminating light that may be associated with the detected, task(s) being carried out in first space 30. Although shown as being coupled to door 24 within first space 30 of environment 10, it is understood that third sensor 120C may be positioned anywhere within first space 30 so long as third sensor 120C is capable of that a task(s) is being carried out within first space 30.

The number of sensors 112, 118, 120A, 120B, 120C included within control system 109 for first space 30, as shown in the non-limiting example of FIG. 1, is understood to be illustrative. As such, although control system 109 of disinfecting light system 100 is shown to include five sensors 112, 118, 120A, 120B, 120C within first space 30, it is understood that control system 109 may include more or less sensors for providing data and/or information to controller 110. Additionally, although first space 30 is shown to include five sensors 112, 118, 120A, 120B, 120C, it is understood that controller 110 may adjust the amount of illuminating light 106 and/or disinfecting energy 108 provided to space 30 by adjusting the output of first disinfecting light fixture 102 based on only a portion of the five sensors 112, 118, 120A, 120B, 120C. In one non-limiting example, control system 109 may include first sensor 112 and one or more third sensors 120A, 120B, 120C. In another non-limiting example, control system 109 may include first sensor 112 and second sensor 118. In an additional non-limiting example, control system 109 may include second sensor 118 and one or more third sensors 120A, 120B, 120C.

Furthermore, although discussed herein as being positioned and/or included within space(s) 30, 32 of environment 10, it is understood that some of sensors 112, 118, 120A, 120B, 120C may be positioned outside of space(s) 30, 32, when applicable. Additionally where sensors 112, 118, 120A, 120B, 120C are positioned outside of space(s) 30, 32, control system 109 may utilize additional components to aid in the measuring, sensing, and/or detected of characteristics relating to space(s) 30, 32, as discussed herein. For example, third sensor 120A configured to detect an occupancy level of space(s) 30, 32 of environment 10 may be configured as a video surveillance system that may monitor activity within space(s) 30, 32. In this non-limiting example, the third sensor 120A configured as video surveillance system may be positioned within a centralized location, outside of space(s) 30, 32 where environment includes a plurality of spaces and/or a plurality of rooms, each including at least one defined space. Additionally in this non-limiting example where third sensor 120A is not positioned within space(s) 30, 32, a video camera or image processing component may be utilized to provide data to third sensor 120A regarding the occupancy level, which in turn may be provided to controller 110 of control system 109, as similarly discussed herein.

Additionally, although discussed herein as sensors 112, 118, 120A, 120B, 120C providing or transmitting data and/or information relating to disinfecting light system 100 and/or space(s) 30, 32 to controller 110, it is understood that some of the data may be provided from distinct components within disinfecting light system 100. For example, data and/or information relating to an amount of illuminating light 106 and/or disinfecting energy 108 provided to first space 30 by first disinfecting light fixture 102 may be provided to controller 110 by first disinfecting light fixture 102 of disinfecting light system 100. That is, first disinfecting light fixture 102 of disinfecting light system 100 may provide data and/or information relating to an amount of illuminating light 106 and/or disinfecting energy 108 to controller 110 in addition to or in place of first sensor 112. In this non-limiting example, controller 110 may adjust illuminating light 106 and/or disinfecting energy 108 provided to first space 30 based on, at least in part, the data and/or information provided to controller 110 directly from first disinfecting light fixture 102.

In the non-limiting example shown in FIG. 1, control system 109 of disinfecting light system 100 may also include at least one access control component 126. As shown in FIG. 1, access control component 126 may be positioned within first space 30 of environment 10. Specifically, access control component 126 may be positioned on wall 42, adjacent door 22, within first space 30. Access control component 126 may be operably coupled to and/or in communication with controller 110 for providing data, information, and/or input to controller for controlling the operation of disinfecting light fixture(s) 102 and more specifically, regulating illuminating light 106 and disinfecting energy 108 provided to first space 30 of environment 10. For example, access control component 126 may provide an override selector or option that may be configured to temporarily permit switching controller 110 off and/or suspending the operational processes performed by controller 110 of control system 109. In this non-limiting example, a user(s) may utilize access control component 126 to manually adjust the operation of controller 110 for controlling illuminating light 106 and/or disinfecting energy 108 provided to first space 30 by first disinfecting light fixture 102, independent of the data provided to controller 110 by sensors 112, 118, 120A, 120B, 120C. As discussed herein, overriding the operation of controller 110 may result in first disinfecting light fixture 102 maintaining a continuous disinfecting energy 108 within first space 30, or alternatively, maintaining an average disinfection level or amount of disinfecting energy 108 in first space 30 at a minimum level. The continuous disinfecting energy 108 or average disinfection leel of disinfecting energy 108 may be maintained despite changes in first space 30 objects or items (e.g., moving chair 24), changes in first space 30 characteristics (e.g., changing wall 18 paint reflectivity, opening/closing curtains on window 12), and/or detected and/or sensed data (e.g., change in bacterial load sensed by second sensor 118, occupancy level detected by third sensor 120A).

In an additional non-limiting example, access control component 126 may include a security access system to allow users access to first space 30 of environment 10. In the non-limiting example, a code associated with a user(s), such as an input code or keycard, may be input, detected, and/or registered with access control component 126, and may provide information, data and/or input from access control component 126 to controller 110 relating to first space 30. For example, when a user inputs their code in access control component 126, access control component 126 may provide information or data relating to an occupancy level of first space 30 based on the user's accessing first space 30 to controller 110. In another example, user(s) may be associated with a specific task to be performed within first space 30. As discussed herein, the specific task associated with the user to be performed in the first space 30 may require a predetermined amount of illuminating light 106 and/or disinfecting energy 108 to be provided to first space 30 by first disinfecting light fixture 102 when performing the task. As such, when user inputs their code in or provides an access key to access control component 126, access control component 126 may provide the user information and/or data to controller 110, which may include the specific task associated with the user, and controller 110 may adjust illuminating light 106 and/or disinfecting energy 108 accordingly, as discussed herein.

In a further non-limiting example, access control component 126 may include and/or be formed as an operational schedule system for first space 30 of environment 10. More specifically, access control component 126 may include and/or be formed as an operational schedule system and/or a system capable of providing a predetermined, operational schedule to controller 110 for controlling the operation of first disinfecting light fixture 102 and/or adjusting illuminating light 106 and/or disinfecting energy 108 provided to first space 30 by first disinfecting light fixture 102. The predetermined, operational schedule provided to controller 110 from access control component 126 may be defined or created by a user(s) and/or operator of disinfecting light system 100 (e.g., building owner or maintenance person for the building include the room forming environment 10). Additionally, the predetermined operational schedule, which determines how controller 110 adjusts illuminating light 106 and/or disinfecting energy 108 to be provided to first space 30, may be based on a plurality of data, factors, information, and/or operational scenarios surrounding the operation of disinfecting light system 100. For example, the predetermined operational schedule provided to controller 110 may be defined and/or created based on a time of day and/or a day in a week. That is, controller 110 may adjust illuminating light 106 and/or disinfecting energy 108 provided to first space 30 by first disinfecting light fixture 102 based on the time of day (e.g., day vs. night), and/or the day in the week (e.g., weekday vs. weekend). In this example, controller 110 may adjust illuminating light 106 and/or disinfecting energy 108 based on the predetermined operational schedule provided by access control component 126 to maintain appropriate illuminating light 106 when first space 30 is in use, and maintain a minimum dosage of disinfecting energy 108 with first space 30 (e.g., at night when first space 30 is not being used). As discussed herein, by adjusting the amount of disinfecting energy 108 when first space 30 is not occupied, an average amount of disinfecting energy 108 (e.g., daily joule dosage) can be maintained over a predetermined period of time while also maintaining illuminating light 106 as needed for use of first space 30.

In another example, the predetermined operational schedule provided to controller 110 may be defined and/or created based on the cost of electricity for operating first disinfecting light fixture 102. As such, controller 110 may adjust illuminating light 106 and/or disinfecting energy 108 provided to first space 30 by first disinfecting light fixture 102 based on when electricity consumption for operating first disinfecting light fixture is at its highest (e.g., peak hours) and its lowest (e.g., off-peak hours). In this example, controller 110 may control operation and/or adjust first disinfecting light fixture 102 to operate (e.g., provide disinfecting energy 108) at minimal power consumption when the electricity costs the most (e.g., peak hours), followed by increased operation (e.g., disinfecting energy 108) when electricity costs the least (e.g., off-peak hours), to maintain an average amount of disinfecting energy 108 (e.g., daily joule dosage) within first space 30.

Although discussed herein with respect to first space 30, and the sensors 112, 118, 120A, 120B, 120C of control system 109 positioned within first space 30, it is understood that the components of disinfecting light system 100 may function and/or operate substantially similar within second space 32. That is, and as shown in the non-limiting example of FIG. 1, second space 32 may include second disinfecting light fixture 104 which may be configured to provide illuminating light 106 and/or disinfecting energy 108 to second space 32, and more specifically workstation 26 included within second space 32. Additionally, and as discussed herein, second disinfecting light fixture 104 may be operably coupled to controller 110 of control system 109. As similarly discussed herein with respect to first disinfecting light fixture 102 and first space 30, controller 110 may receive data from fourth sensor 122 and fifth sensor 124 included within second space 32 (e.g., positioned on workstation 26), and adjust illuminating light 106 and/or disinfecting energy 108 provided to second space 32 by second disinfecting light fixture 104. Fourth sensor 122 and fifth sensor 124 positioned within second space 32 may be substantially similar to first sensor 112 and second sensor 118, respectively, positioned within first space 30. That is, fourth sensor 122 may be any suitable sensor capable of measuring an amount of disinfecting energy 108 provided to second space 32 by second disinfecting light fixture 104 of disinfecting light system 100. Additionally, fifth sensor 124 may be any suitable sensor capable of sensing bacterial load, bioburden, and/or microbial load within second space 32 of environment 10. Redundant explanation of these components has been omitted for clarity.

In the non-limiting example shown in FIG. 1, second space 32 may not include any third sensors 120A, 120B, 120C configured as environmental characteristic sensors, and/or may be sensors configured to measure or detect environmental characteristics of second space 32 of environment 10. However, because second space 32 is included within first space 30, controller 110, configured to adjust illuminating light 106 and/or disinfecting energy 108 provided to second space 32 by second disinfecting light fixture 104, may utilize environmental characteristic data measured, obtained, sensed, and/or identified by third sensors 120A, 120B, 120C positioned within first space 30. That is, the information obtained by third sensors 120A, 120B, 120C and provided to controller 110 for adjusting illuminating light 106 and/or disinfecting energy 108 provided to first space 30 by first disinfecting light fixture 102 may also be utilized to by controller 110 to adjust illuminating light 106 and/or disinfecting energy 108 provided to second space 32 by second disinfecting light fixture 104. This may be because environmental characteristics that apply to first space 30 (e.g., occupancy level) may also affect and/or be the same for second space 32. In another non-limiting example (not shown), second space 32 may include at least one additional sixth sensor that is substantially similar to third sensors 120A, 120B, 120C of first space 30. That is, the sixth sensor(s) included within second space 32 may be configured as environmental characteristic sensors, and/or may be sensors configured to measure or detect environmental characteristics of second space 32 of environment 10.

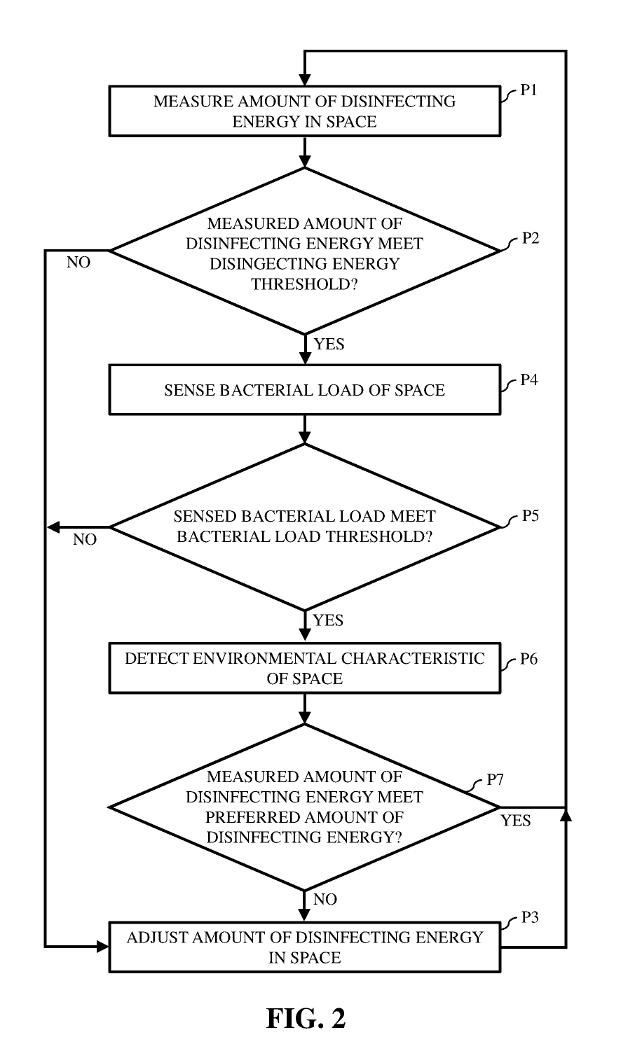

FIG. 2 shows a flow diagram illustrating non-limiting example processes of regulating disinfecting energy 108 (as well as illuminating light 106) generated by a disinfecting light system 100 within space(s) 30, 32 of environment 10. These processes can be performed, e.g., by at least one controller 110 of control system 109 for disinfecting light system 100 (see, FIG. 1), as described herein. In other cases, these processes can be performed according to a computer-implemented method of regulating disinfecting energy 108 generated by a disinfecting light system 100 within space(s) 30, 32 of environment 10. In still other embodiments, these processes can be performed by executing computer program code on a computing device(s), causing the computing device(s), and specifically controller 110, to regulate disinfecting energy 108 generated by a disinfecting light system 100 within space(s) 30, 32 of environment 10. The processes shown in the flow diagram of FIG. 2 are discussed in detail below.

In process P1, an amount of disinfecting energy in a space of an environment may be measured. Specifically, an amount of disinfecting energy provided to the space of the environment by a disinfecting light fixture of a disinfecting light system may be measured, sensed, detected and/or determined. The measured amount of energy provided to the space of the environment by the disinfecting light fixture of the disinfecting light system may be measured by at least one sensor positioned within the space receiving the disinfecting energy.

In process P2, the measured amount of disinfecting energy provided to the space of the environment may be compared to a predetermined disinfecting energy threshold to determine if the measured amount of disinfecting energy meets the disinfecting energy threshold. Specifically, it may be determined if the measured amount of disinfecting energy, provided to the space by the disinfecting light fixture of the disinfecting light system and measured by the sensor(s) within the space, meets the disinfecting energy threshold. The predetermined disinfecting energy threshold may be a predetermined or desired amount, level, minimum, and/or range of disinfecting energy to be provided to the space. That is, the predetermined disinfecting energy threshold may be based on maintaining the predetermined or desired amount, level, minimum, and/or range of disinfecting energy to be provided to the space. The predetermined or desired amount, level, minimum, and/or range of disinfecting energy to be provided to the space may be an instant or real-time desired amount of disinfecting energy, or alternatively, may be a desired amount of disinfecting energy provided over a predetermined period of time (e.g., daily dosage). In another non-limiting example, the predetermined disinfecting energy threshold may be configured to maintain a predetermined average disinfection amount, level and/or range of the disinfecting energy provided to space over a predetermined time.

In a non-limiting example, the measured amount of disinfecting energy meets the disinfecting energy threshold when it is equal to or within the range of the desired, disinfecting energy to be provided to the space. The controller of the control system for the disinfecting light system may determine if the measured amount of disinfecting energy meets the disinfecting energy threshold. If it is determined that the measured amount of disinfecting energy does not meet the disinfecting energy threshold (e.g., “NO” at process P2), the processes may proceed to process P3. Conversely, if it is determined that the measured amount of disinfecting energy does meet the disinfecting energy threshold (e.g., “YES” at process P2), the processes may proceed to process P4.

In response to determining the measured amount of disinfecting energy does not meet the disinfecting energy threshold (e.g., “NO” at process P2), the amount of disinfecting energy in the space may be adjusted in process P3. That is, in process P3, the amount of disinfecting energy provided to the space by the disinfecting light fixture of the disinfecting light system may be adjusted, changed, and/or altered. The amount of disinfecting energy provided to the space may be adjusted by altering the operation of the disinfecting light fixture to one of increase or decrease the amount of disinfecting energy generated by the disinfecting light fixture and provided to the space. In a non-limiting example, the controller of the control system for the disinfecting light system may be operably coupled to the disinfecting light fixture and may be configured to control the operation of disinfecting light fixture to adjust (e.g., increase, decrease) the amount of disinfecting energy generated by the disinfecting light fixture. The amount of disinfecting energy provided to the space may be adjusted by the controller of the control system to maintain a predetermined or desired amount, level, minimum, and/or range of disinfecting energy to be provided to the space over a predetermined period of time. Additionally, or alternatively, adjusting the amount of disinfecting energy provided to the space may include maintaining a predetermined average disinfection amount, level, and/or range of the disinfecting energy provided to space over a predetermined time. Furthermore, the amount of disinfecting energy generated by the disinfecting light fixture can be altered or adjusted by increasing or decreasing the brightness or dynamically changing the violet content of the illuminating light provided to the space by the disinfecting light fixture.

In a non-limiting example where the measured amount of disinfecting energy is less than the disinfecting energy threshold, the controller may increase the amount of disinfecting energy provided to the space by the disinfecting light fixture until the measured amount of disinfecting energy meets the disinfecting energy threshold. Additionally in a non-limiting example where the measured amount of disinfecting energy is greater than the disinfecting energy threshold, the controller may decrease or maintain the amount of disinfecting energy provided to the space by the disinfecting light fixture until the measured amount of disinfecting energy meets the disinfecting energy threshold. Alternatively in the non-limiting example where the measured amount of disinfecting energy is greater than the disinfecting energy threshold, the controller may stop the disinfecting light fixture from generating and providing disinfecting energy to the space until the measured amount of disinfecting energy meets the disinfecting energy threshold.

In response to determining the measured amount of disinfecting energy meets the disinfecting energy threshold (e.g., “YES” at process P2), a bacterial load of the space may be sensed in process P4. Specifically, a bacterial load of the space of the environment may be sensed, measured, detected, and/or determined. The sensed bacterial load of the space of the environment may be sensed and/or detected by at least one sensor positioned within the space. In another non-limiting example, the sensed bacterial load of the space of the environment may be determined and/or calculated using a correlated measurement.

In process P5, the sensed bacterial load of the space may be compared to a bacterial load threshold to determine if the sensed bacterial load meets the bacterial load threshold. Specifically, it may be determined if the sensed bacterial load for the space, as detected or sensed by the sensor(s) within the space, meets the predetermined bacterial load threshold. The predetermined, bacterial load threshold may be a predetermined or desired amount, level, maximum, and/or range for an acceptable bacterial load of the space. That is, the predetermined bacterial load threshold for the space may be based on maintaining the predetermined or desired amount, level, maximum, and/or range of bacterial load within the space. In another non-limiting example, the predetermined bacterial threshold may be based on maintaining a predetermined average bacterial load amount, level, and/or range of the space over a predetermined time.

In a non-limiting example, the sensed bacterial load meets the predetermined bacterial load threshold when it is equal to or within the range of the desired, bacterial load for the space. The controller of the control system for the disinfecting light system may determine if the sensed bacterial load meets the bacterial load threshold. If it is determined that the sensed bacterial load does not meet the bacterial load threshold (e.g., “NO” at process P5), the processes may proceed to process P3. Conversely, if it is determined that the sensed bacterial load does meet the bacterial load threshold (e.g., “YES” at process P5), the processes may proceed to process P6.

In response to determining the sensed bacterial load does not meet the bacterial load threshold (e.g., “NO” at process P5), the amount of disinfecting energy in the space may be adjusted in process P3. That is, in process P3, the amount of disinfecting energy provided to the space by the disinfecting light fixture of the disinfecting light system may be adjusted, changed, and/or altered. As similarly discussed herein, the controller of the control system for the disinfecting light system may be operably coupled to the disinfecting light fixture and may be configured to control the operation of disinfecting light fixture to adjust (e.g., increase, decrease) the amount of disinfecting energy generated by the disinfecting light fixture and provided to the space. As discussed herein, the disinfecting energy generated by the disinfecting light fixture may alter, adjust, and/or control the bacterial load, bioburden, and/or microbial load within the space receiving the disinfecting energy. In a non-limiting example where the sensed bacterial load is greater than the bacterial load threshold, the controller may increase the amount of disinfecting energy provided to the space by the disinfecting light fixture until the sensed bacterial load meets the bacterial load threshold. Additionally in a non-limiting example where the sensed bacterial load is less than the bacterial load threshold, the controller may decrease or maintain the amount of disinfecting energy provided to the space by the disinfecting light fixture until the sensed bacterial load meets the bacterial load threshold. Alternatively in the non-limiting example where the sensed bacterial load is less than the bacterial load threshold, the controller may stop the disinfecting light fixture from generating the disinfecting energy until the sensed bacterial load meets the bacterial load threshold.

In response to determining the sensed bacterial load meets the bacterial load threshold (e.g., “YES” at process P5), an environmental characteristic(s) of the space may be detected in process P6. Specifically in process P6, an environmental characteristic(s) related to and/or associated with the space including the disinfecting light system may be detected. The environmental characteristic(s) of the space may be sensed and/or detected by at least one sensor positioned within the space. For example, the detected environmental characteristic may include and/or be based upon an occupancy level of the space (e.g., if the space is occupied, the number of users that may occupy the space, a change in user-occupancy for the space) being provided the disinfecting energy by the disinfecting light fixture. In another non-limiting example, the detected environmental characteristic may include and/or be based upon an amount of natural light in the space, and/or a predetermined amount of natural disinfecting energy associated with and/or included within or provided with the natural light. In an additional non-limiting example, the detected environmental characteristic may include and/or be based upon at least one task being carried out in the space.

In addition to detecting the environmental characteristic(s) in process P6, a preferred amount of disinfecting energy associated with the detected environmental characteristic(s) may be identified. That is, detecting the environmental characteristic(s) in process P6 may also include identifying a preferred amount of disinfecting energy associated with detected environmental characteristics that may be provided to the space by the disinfecting light fixture of the disinfecting light system. The preferred amount of disinfecting energy associated with the detected environmental characteristic(s) may be predefined and/or predetermined based on the environmental characteristic(s) and/or characteristics of the space provided the disinfecting energy. In non-limiting examples, the preferred amount of disinfecting energy associated with the detected environmental characteristic(s) may be stored on the controller of the control system, or may be provided to the controller from an external source (e.g., storage device), such that when the detected environmental characteristic(s) is provided to the controller of the control system, the preferred amount of disinfecting energy associated with detected environmental characteristics may also be provided to and/or recognized by the controller.

The preferred amount of disinfecting energy associated with detected environmental characteristics may be based on, related to, and/or associated with the space provided the disinfecting light. For example, the preferred amount of disinfecting energy associated with the detected occupancy level of the space may include various preferred amounts of disinfecting energy based upon distinct occupancy levels of the space. In another non-limiting example, the preferred amount of disinfecting energy associated with the detected natural light in the space and/or the amount of natural disinfecting energy associated with the natural light, may include various preferred amounts of disinfecting energy based upon the amount of natural light and/or natural disinfecting energy in the space. In an additional non-limiting example, the preferred amount of disinfecting energy associated with the detected task carried out in the space may include various preferred amounts of disinfecting energy based upon various tasks being carried out in the space.

In process P7, it may be determined if the measured amount of disinfecting energy provided to the space by the disinfecting light system meets the preferred amount of disinfecting energy associated with the detected, environmental characteristic(s). Specifically, it may be determined if the measured amount of disinfecting energy provided to the space by the disinfecting light system (e.g., process P1) meets the preferred amount of disinfecting energy associated with the detected, environmental characteristic(s) (e.g., process P6). In a non-limiting example, the measured amount of disinfecting energy provided to the space by the disinfecting light system meets the preferred amount of disinfecting energy associated with the detected, environmental characteristic(s) when it is equal to or within the range of the preferred amount of disinfecting energy associated with the detected, environmental characteristic. The controller of the control system for the disinfecting light system may determine if the measured amount of disinfecting energy provided to the space by the disinfecting light system meets the preferred amount of disinfecting energy associated with the detected, environmental characteristic(s). If it is determined that the measured amount of disinfecting energy provided to the space does not meet the preferred amount of disinfecting energy associated with the detected, environmental characteristic(s) (e.g., “NO” at process P7), the processes may proceed to process P3. Conversely, if it is determined that the measured amount of disinfecting energy provided to the space does meet the preferred amount of disinfecting energy associated with the detected, environmental characteristic(s) (e.g., “YES” at process P7), the processes repeat and/or may proceed back to process P1 and may begin again.

In response to determining the measured amount of disinfecting energy provided to the space does not meet the preferred amount of disinfecting energy associated with the detected, environmental characteristic(s) (e.g., “NO” at process P6), the amount of disinfecting energy in the space may be adjusted in process P3. That is, in process P3, the amount of disinfecting energy provided to the space by the disinfecting light fixture of the disinfecting light system may be adjusted, changed, and/or altered. The amount of disinfecting energy provided to the space may be adjusted by altering the operation of the disinfecting light fixture to one of increase or decrease the amount of disinfecting energy generated by the disinfecting light fixture and provided to the space. In a non-limiting example, the controller of the control system for the disinfecting light system may be operably coupled to the disinfecting light fixture and may be configured to control the operation of disinfecting light fixture to adjust (e.g., increase, decrease) the amount of disinfecting energy generated by the disinfecting light fixture. In a non-limiting example where the measured amount of disinfecting energy is less than the preferred amount of disinfecting energy associated with the detected, environmental characteristic(s) (e.g., occupancy level, natural light, natural disinfecting energy, task(s)), the controller may increase the amount of disinfecting energy provided to the space by the disinfecting light fixture until the measured amount of disinfecting energy meets the preferred amount of disinfecting energy. Additionally in a non-limiting example where the measured amount of disinfecting energy is greater than the preferred amount of disinfecting energy associated with the detected, environmental characteristic(s) (e.g., occupancy level, natural light, natural disinfecting energy, task(s)), the controller may decrease or maintain the amount of disinfecting energy provided to the space by the disinfecting light fixture until the measured amount of disinfecting energy meets the preferred amount of disinfecting energy. Alternatively in the non-limiting example where the measured amount of disinfecting energy is greater than the preferred amount of disinfecting energy associated with the detected, environmental characteristic(s), the controller may stop the disinfecting light fixture from generating and providing disinfecting energy to the space until the measured amount of disinfecting energy meets the preferred amount of disinfecting energy.

Although shown in succession, it is understood that some of the processes illustrated in FIG. 2 for regulating the disinfecting energy generated by the disinfecting light system may be performed concurrently. For example, processes P1 and P4 may be performed concurrently, and subsequent processes P2 and P5 may also be performed concurrently after performing processes P1 and P4. Additionally, it is understood that the order in which at least some of the processes of FIG. 2 for regulating the disinfecting energy are performed is illustrative. As such, some of the processes may be performed in a distinct order than that shown in the non-limiting example of FIG. 2. For example, processes P4 and P5 may be performed prior to performing processes P1 and P2. Additionally, or alternatively, processes P6 and P7 may be performed prior to performing processes P4 and P5.

Additionally, the processes for regulating the disinfecting energy generated by the disinfecting light system may be performed independent of the operation and/or adjustment of the illuminating light generated by the disinfecting light system. That is, regulating the disinfecting energy by adjusting the amount of disinfecting energy provided to the space by the disinfecting light fixture may be performed independent of adjusting the amount of the illuminating light. The controller of the control system may adjust the amount of the illuminating light provided to space by the disinfecting light fixture based on the preferred amount of illuminating light that may be associated with the detected, environmental characteristics of the space.

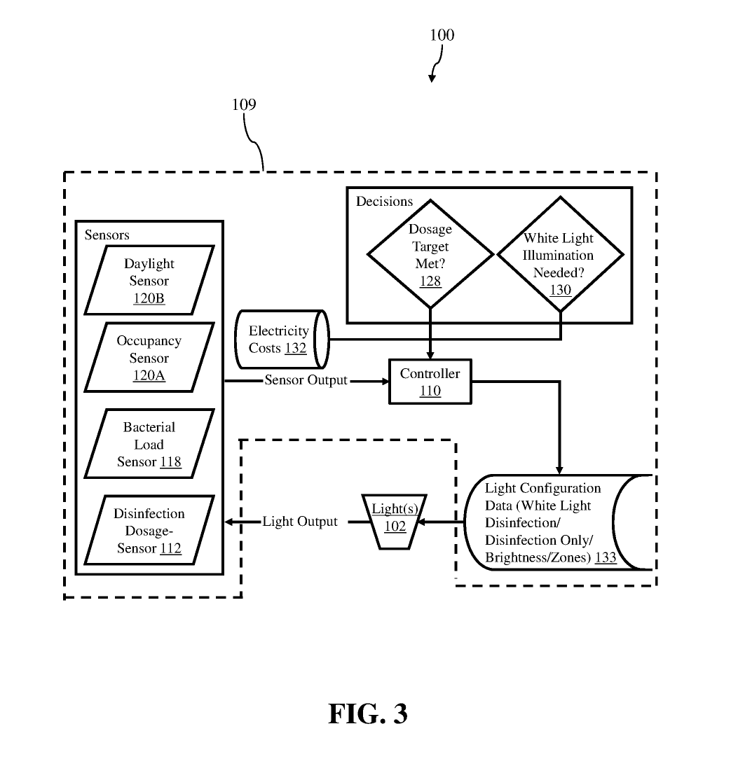

Additionally in other non-limiting examples, the controller of the control system for the disinfecting light system may receive and process a variety of data and/or information from various sources (e.g., sensors, storage devices, and the like) before regulating the disinfecting energy. That is, the controller may receive and process a variety of distinct data and/or information, from various sources, before adjusting the amount of disinfecting energy generated by the disinfecting light fixture. Turning to FIG. 3, a non-limiting schematic view of disinfecting light system 100 including control system 109 is shown. Specifically, FIG. 3 shows a schematic view of disinfecting light system 100 including control system 109, and a flow process of data and/or information through the various components of disinfecting light system 100 and/or control system 109. It is understood that similarly numbered and/or named components may function in a substantially similar fashion. Redundant explanation of these components has been omitted for clarity.

In the non-limiting example shown in FIG. 3, a plurality of sensors 112, 118, 120A, 120B of control system 109 may provide sensor output and/or data to controller 110 for aiding in the control of the operation of disinfecting light fixture 102 of disinfecting light system 100. In the non-limiting example, and as discussed herein with respect to FIG. 1, first sensor 112 may provide data and/or information relating to a measured amount of disinfecting energy 108 provided to a space, for example first space 30, to controller 110. Additionally, second sensor 118 may provide data and/or information relating to a sensed bacterial load of first space 30, third sensor 120A may provide data and/or information relating to a detected occupancy level of first space 30, and third sensor 120B may provide data and/or information relating to an amount of natural light 20 and/or amount of natural disinfecting energy 40 (see, FIG. 1) provided to first space 30.

Additionally, controller 110 may receive additional information and/or data from external sources and/or components, such as external storage devices, to aid in the control aiding in the control of the operation of disinfecting light fixture 102 of disinfecting light system 100. For example, and as shown in FIG. 3, a disinfecting energy target component 128 may provide data and/or information which aids controller 110 in determining if disinfecting energy 108 provided to first space 30 by first disinfecting light fixture 102 needs to be adjusted. That is, disinfecting energy target component 128 may provide data and/or information to determine if the amount of disinfecting energy 108 provided to first space 30, and measured by first sensor 112, is equal to or within the range of a predetermined or desired amount, level, minimum, and/or range of disinfecting energy 108 to be provided to first space 30, as discussed herein. In one non-limiting example, disinfecting energy target component 128 may include data or information relating to the predetermined disinfecting energy threshold, similarly discussed herein with respect to processes P1 and P2 of FIG. 2. Additionally in another non-limiting example, disinfecting energy target component 128 may include data or information relating to the preferred amount of disinfecting energy associated with detected environmental characteristics detected in first space 30 (e.g., occupancy level, amount of natural light, amount of natural disinfecting energy, task(s)), similarly discussed herein with respect to processes P6 and P7 of FIG. 2. Additionally, disinfecting energy target component 128 may also be configured to prompt controller 110 to determine if a dosage target for disinfecting energy 108 is met. That is, disinfecting energy target component 128 may also be configured to prompt controller 110 to determine if the amount of disinfecting energy 108 provided to first space 30, and measured by first sensor 112, is equal to or within the range of a predetermined or desired amount, level, minimum, and/or range of disinfecting energy 108 to be provided to first space 30.

Also shown in the non-limiting example of FIG. 3, an illuminating light target component 130 may provide data and/or information which aids controller 110 in determining if illuminating light 106 provided to first space 30 by first disinfecting light fixture 102 needs to be adjusted. In one non-limiting example, illuminating light target component 130 may include data or information relating to a predetermined illuminating light threshold, which may be based on the range of a predetermined or desired amount, level, minimum, and/or range of illuminating light 106 to be provided to first space 30. Additionally in another non-limiting example, illuminating light target component 130 may include data or information relating to the preferred amount of illuminating light associated with detected environmental characteristics detected in first space 30 (e.g., occupancy level, amount of natural light, natural disinfecting energy, task(s)), similarly discussed herein with respect to third sensors 120A, 120B, 120C of FIG. 1. As such, illuminating light target component 130 may provide data and/or information to controller 110 to determine if the amount of illuminating light 106 provided to first space 30 is equal to or within the range of the predetermined amount or range of illuminating light 106, or alternatively the preferred amount of illuminating light, to be provided to first space 30, as discussed herein. Additionally, illuminating light target component 130 may also be configured to prompt controller 110 to determine if white light illumination (e.g., illuminating light 106) is needed within first space 30. That is, illuminating light target component 130 may also be configured to prompt controller 110 to determine if the amount of illuminating light 106 provided to first space 30 is equal to or within the range of the predetermined amount or range of illuminating light, or preferred amount of illuminating light associated with detected environmental characteristics, to be provided to first space 30.

Illuminating light target component 130 may also be in communication with and/or may receive data from an additional source or component before providing data and/or prompting controller 110, as discussed herein. For example, and as shown in FIG. 3, an electrical cost storage device 132 may be operably coupled to and/or in communication with illuminating light target component 130. Electrical cost storage device 132 may include data and/or information relating to the cost of electricity for operating first disinfecting light fixture 102 of disinfecting light system 100 (e.g., peak hours, off-peak hours). As such, when prompting controller 110 to determine if white light illumination (e.g., illuminating light 106) is needed within first space 30, illuminating light target component 130 may also provide and/or consider information relating the cost of electricity for operating first disinfecting light fixture 102 provided by electrical cost storage device 132. Although shown as being operably coupled to and/or in communication with illuminating light target component 130, it is understood that electrical cost storage device 132 may also be operably coupled to and/or in communication with disinfecting energy target component 128. In this non-limiting example, when prompting controller 110 to determine if a dosage target for disinfecting energy 108 is met, disinfecting energy target component 128 may also provide and/or consider information relating the cost of electricity for operating first disinfecting light fixture 102 provided by electrical cost storage device 132.

Additionally in the non-limiting example shown in FIG. 3, after controller 110 determines that the amount of illuminating light 106 and/or disinfecting energy 108 provided to space 30 may require adjustment, controller 110 may provide input (e.g., a signal) to a light configuration data component 133, which may aid controller 110. That is, light configuration data component 133 may include data and/or information relating to the operation and/or function of first disinfecting light fixture 102 of disinfecting light system 100. As such, when controller 110 determines that the amount of illuminating light 106 and/or disinfecting energy 108 provided to space 30 requires adjustment, controller 110 may utilize light configuration data component 133, including data and/or information relating to the operation and/or function of first disinfecting light fixture 102, to control the operation of first disinfecting light fixture 102. Controller 110 may utilize light configuration data component 133 to ensure that the amount of illuminating light 106 and/or disinfecting energy 108 is adjusted, such that the amount of illuminating light 106 and/or disinfecting energy 108 is equal to or within the range of the desired (e.g., predetermined or preferred) amount of illuminating light 106 and/or disinfecting energy 108, as discussed herein.

FIG. 4 depicts a schematic view of control system 109, and the various components included within control system 109. In the non-limiting example shown in FIG. 4, control system 109 may include at least one controller 110 that may be configured to aid in regulating disinfecting energy 108 generated by disinfecting light system 100 within space(s) 30, 32 by performing the processes P1-P7 discussed herein with respect to FIG. 2. Controller(s) 110 shown in FIG. 4 may be substantially similar to controller 110 discussed herein with respect to FIGS. 1 and/or 3. It is understood that similarly numbered and/or named components may function in a substantially similar fashion. Redundant explanation of these components has been omitted for clarity.

It is understood that controller(s) 110 may be implemented as a computer program product stored on a computer readable storage medium. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Python, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Control system 109 may include any type of controller(s) 110, which may include, for example, at least one processor 134, storage component 136, input/output (I/O) component(s) 138 (including users electronic devices discussed herein), and a communications pathway 140. In general, processor(s) 134 execute program code which is at least partially fixed in storage component 136. While executing program code, processor(s) 134 can process data, which can result in reading and/or writing transformed data from/to storage component 136 and/or I/O component(s) 138 for further processing. The pathway 140 provides a communications link between each of the components in controller(s) 110. I/O component 138 can comprise one or more human I/O devices, which enables user(s) 142 to interact with controller(s) 110. Controller(s) 110 may also be implemented in a distributed manner such that different components reside in different physical locations.

Storage component 136 may also include modules, data and/or electronic information relating to various other aspects of control system 109. Specifically, operational modules, information, and/or data relating to disinfecting light system data 144, disinfecting energy data 146, bacterial load data 148, environmental characteristic data 150, space data 152, task data 154, and schedule data 156. The operational modules and/or data may include the required information and/or may allow control system 109, and specifically controller 110, to perform the processes discussed herein for regulating disinfecting energy 108 generated by disinfecting light system 100 within space(s) 30, 32. Additionally, sensors 112, 118, 120A, 120B, 120C, 122, 124 may in communication with control system 109, and more specifically controller 110 of control system 109, to transmit measured, sensed, and/or detected data (e.g., sensed bacterial load, measure amount of disinfecting light in space(s) 30, 32, and the like) to controller 110. Furthermore, controller 110 may utilize the transmitted data from sensors 112, 118, 120A, 120B, 120C, 122, 124, and the operational modules, information, and/or data stored on storage component 136 (e.g., disinfecting light system data 144, disinfecting energy data 146, bacterial load data 148, and so on) to regulate disinfecting energy 108 generated by disinfecting light system 100 within space(s) 30, 32, as discussed herein.

Control system 109, and specifically controller 110 of control system 109, may also be in communication with an external storage component 158. External storage component 158 may be configured to store various modules, data and/or electronic information relating to various other aspects of control system 109, similar to storage component 136 of controller(s) 110. Additionally, external storage component 158 may be configured to share (e.g., send and receive) data and/or electronic information with controller(s) 110 of control system 109. In the non-limiting example shown in FIG. 4, external storage component 158 may include any or all of the operational modules and/or data shown to be stored on storage component 136 (e.g., data 144-156). In a non-limiting example, external storage component 158 may be a cloud-based storage component or system.

Furthermore, it is understood that controller(s) 110 of control system 109 or relevant components thereof (such as an API component, agents, etc.) may also be automatically or semi-automatically deployed into a computer system by sending the components to a central server or a group of central servers. The components are then downloaded into a target computer that will execute the components. The components are then either detached to a directory or loaded into a directory that executes a program that detaches the components into a directory. Another alternative is to send the components directly to a directory on a client computer hard drive. When there are proxy servers, the process will select the proxy server code, determine on which computers to place the proxy servers' code, transmit the proxy server code, and then install the proxy server code on the proxy computer. The components will be transmitted to the proxy server and then it will be stored on the proxy server.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. “Approximately” as applied to a particular value of a range applies to both values, and unless otherwise dependent on the precision of the instrument measuring the value, may indicate +/−10% of the stated value(s).

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A control system for a disinfecting light system, the control system comprising:
a first sensor positioned within a space illuminated by the disinfecting light system, the first sensor measuring an amount of disinfecting energy provided to the space by the disinfecting light system;
a second sensor positioned within the space illuminated by the disinfecting light system, the second sensor detecting an environmental characteristic of the space; and
a controller operably coupled to the first sensor and the second sensor, the controller regulating the disinfecting energy generated by the disinfecting light system by performing processes including:
adjusting the amount of disinfecting energy provided to the space by the disinfecting light system in response to at least one of:
the amount of disinfecting energy provided to the space by the disinfecting light system measured by the first sensor, or
the environmental characteristic detected by the second sensor.

2. The control system of claim 1, wherein the controller adjusts the amount of disinfecting energy provided to the space by the disinfecting light system in response to at least one of:
determining the measured amount of disinfecting energy provided to the space by the disinfecting light system does not meet a disinfecting energy threshold, or
determining the measured amount of disinfecting energy provided to the space by the disinfecting light system does not meet a preferred amount of disinfecting energy associated with the detected, environmental characteristic.

3. The control system of claim 1, wherein the first sensor includes at least one of:
a spectrometer,
a photodiode, or
a watt meter.

4. The control system of claim 1, further comprising:
a third sensor positioned within the space illuminated by the disinfecting light system, the third sensor sensing a bacterial load of the space.

5. The control system of claim 4, wherein the controller is operably coupled to the third sensor, and regulates the disinfecting energy generated by the disinfecting light system by performing additional processes including:
adjusting the amount of disinfecting energy provided to the space by the disinfecting light system in response to the bacterial load of the space sensed by the third sensor.

6. The control system of claim 4, wherein the third sensor senses the bacterial load of the space by at least one of:
measuring the bacterial load of air within the space, or
measuring the bacterial load on a surface of an object positioned within the space.

7. The control system of claim 4, wherein the third sensor includes at least one of:
an optical sensor,
an oxygen-depletion sensor, or
a luminometer.

8. The control system of claim 1, wherein the second sensor includes at least one of:
an occupancy sensor measuring an occupancy level of the space,
a daylight sensor sensing an amount of natural light in the space, or
a task-identifying sensor identifying at least one task being carried out in the space.

9. The control system of claim 1, wherein the controller adjusts the amount of disinfecting energy provided to the space by the disinfecting light system by performing processes including:
maintaining a predetermined amount of disinfecting energy within the space over a predetermined period.

10. The control system of claim 1, wherein the controller adjusts the amount of disinfecting energy provided to the space by the disinfecting light system by performing processes including:
maintaining an average amount of the disinfecting energy within the space at a minimum level.

11. A control system for a disinfecting light system, the control system comprising:
a first sensor positioned within a space including the disinfecting light system, the first sensor sensing a bacterial load of the space;
a second sensor positioned within the space including the disinfecting light system, the second sensor detecting an environmental characteristic of the space; and
a controller operably coupled to the first sensor and the second sensor, the controller regulating a disinfecting energy generated by the disinfecting light system by performing processes including:
adjusting the amount of disinfecting energy provided to the space by the disinfecting light system in response to at least one of:
the bacterial load of the space sensed by the first sensor, or
the environmental characteristic detected by the second sensor.

12. The control system of claim 11, wherein the controller adjusts the amount of disinfecting energy provided to the space by the disinfecting light system in response to:
determining the sensed bacterial load of the space does not meet a bacterial load threshold.

13. The control system of claim 11, wherein the first sensor senses the bacterial load of the space by at least one of:
measuring the bacterial load of air within the space, or
measuring the bacterial load on a surface of an object positioned within the space.

14. The control system of claim 11, further comprising:
a third sensor positioned within the space including the disinfecting light system, the third sensor measuring the amount of disinfecting energy provided to the space by the disinfecting light system.

15. The control system of claim 14, wherein the controller is operably coupled to the third sensor, and regulates the disinfecting energy generated by the disinfecting light system by performing additional processes including at least one of:
adjusting the amount of disinfecting energy provided to the space by the disinfecting light system in response to the amount of disinfecting energy provided to the space by the disinfecting light system measured by the third sensor, or
adjusting the amount of disinfecting energy provided to the space by the disinfecting light system in response to the measured amount of disinfecting energy provided to the space by the disinfecting light system not meeting a preferred amount of disinfecting energy associated with the detected, environmental characteristic.

16. The control system of claim 11, wherein the second sensor includes at least one of:
an occupancy sensor measuring an occupancy level of the space,
a daylight sensor sensing an amount of natural light in the space, or
a task-identifying sensor identifying at least one task being carried out in the space.

17. A control system for a disinfecting light system, the control system comprising:
a first sensor positioned within a first space illuminated by a first disinfecting light fixture of the disinfecting light system, the first sensor measuring an amount of disinfecting energy provided to the first space by the first disinfecting light fixture;
a second sensor positioned within the first space illuminated by the first disinfecting light fixture, the second sensor sensing a bacterial load of the first space; and
a controller operably coupled to the first sensor and the second sensor, the controller regulating the disinfecting energy generated by the first disinfecting light fixture by performing processes including:
adjusting the amount of disinfecting energy provided to the first space by the first disinfecting light fixture in response to at least one of:
the amount of disinfecting energy provided to the first space by the first disinfecting light fixture measured by the first sensor, or
the bacterial load of the first space sensed by the second sensor.

18. The control system of claim 17, wherein the second sensor senses the bacterial load of the first space by at least one of:
measuring the bacterial load of air within the first space, or
measuring the bacterial load on a surface of an object positioned within the first space.

19. The control system of claim 17, further comprising:
a third sensor positioned within the first space, the third sensor detecting an environmental characteristic of the first space.

20. The control system of claim 19, wherein the detected environmental characteristic of the first space includes at least one of:
an occupancy level of the first space,
an amount of natural light in the first space, or
at least one task being carried out in the first space.

21. The control system of claim 19, wherein the controller adjusts the amount of disinfecting energy provided to the first space by the first disinfecting light fixture in response to the environmental characteristic of the first spaced detected by the third sensor.

22. The control system of claim 17, further comprising:
a fourth sensor positioned within a second space illuminated by a second disinfecting light fixture of the disinfecting light system, the fourth energy sensor measuring an amount of disinfecting energy provided to the second space by the second disinfecting light fixture; and
a fifth sensor positioned within the second space illuminated by the second disinfecting light fixture, the fifth sensor sensing a bacterial load of the second space,
wherein the controller is operably coupled to the fourth sensor and the fifth sensor, and the controller regulates the disinfecting energy generated by the second disinfecting light fixture by performing processes including:
adjusting the amount of disinfecting energy provided to the second space by the second disinfecting light fixture in response to:
the amount of disinfecting energy provided to the second space by the second disinfecting light fixture measured by the fourth sensor, or
the bacterial load of the second space sensed by the fifth sensor.

23. The control system of claim 22, further comprising:
a sixth sensor positioned within the second space, the sixth sensor detecting an environmental characteristic of the second space.

24. The control system of claim 23, wherein the detected environmental characteristic of the second space includes at least one of:
an occupancy level of the second space,
an amount of natural light in the second space, or
at least one task being carried out in the second space.

25. The control system of claim 23, wherein the controller adjusts the amount of disinfecting energy provided to the second space by the second disinfecting light fixture in response to the environmental characteristic of the second spaced detected by the sixth sensor.

26. The control system of claim 22, wherein the controller adjusts the amount of disinfecting energy provided to the second space by the second disinfecting light fixture in response to the environmental characteristic of the first spaced detected by the third sensor.

27. A method of regulating a disinfecting energy generated by a disinfecting light system, the method comprising:
comparing a measured amount of disinfecting energy provided to a space by the disinfecting light system to a disinfecting energy threshold;
comparing a preferred amount of disinfecting energy associated with a detected, environmental characteristic to the measured amount of disinfecting energy provided to the space by the disinfecting light system; and
adjusting the amount of disinfecting energy provided to the space by the disinfecting light system in response to at least one of:
determining the measured amount of disinfecting energy provided to the space by the disinfecting light system does not meet the disinfecting energy threshold, or
determining the measured amount of disinfecting energy provided to the space by the disinfecting light system does not meet the preferred amount of disinfecting energy associated with the detected, environmental characteristic.

28. The method of claim 27, further comprising:
comparing a sensed bacterial load of the space to a bacterial load threshold; and
adjusting the amount of disinfecting energy provided to the space by the disinfecting light system in response to determining the sensed bacterial load of the space does not meet the bacterial load threshold.

29. The method of claim 27, wherein adjusting the amount of disinfecting energy provided to the space by the disinfecting light system further comprises:
maintaining an average amount of the disinfecting energy within the space at a minimum level.

30. The method of claim 27, wherein adjusting the amount of disinfecting energy provided to the space by the disinfecting light system further comprises:
maintaining a predetermined amount of disinfecting energy within the space over a predetermined period.
